(19) United States
(12) Patent Application Publication
(10) Pub. No.: US 2010/0211408 A1
(43) Pub. Date: Aug. 19, 2010

(54) SYSTEMS AND METHODS FOR GENERATING MEDICAL DIAGNOSES

(76) Inventors: Carl Hyunsuk Park, Philadelphia, PA (US); Jay Leonard Federman, Philadelphia, PA (US); Michael Thomas Trese, Bloomfield Hills, MI (US); Antonio Capone, JR., Birmingham, MI (US); Kimberly Alyson Drenser, Bloomfield, MI (US)

Correspondence Address: FOX ROTHSCHILD LLP, 997 Lenox Drive, Bldg. #3, Lawrenceville, NJ, 08648, US

(21) Appl. No.: 12/372,157

(22) Filed: Feb. 17, 2009

(51) Int. Cl. G06Q 50/00; A61B 3/14

(52) U.S. Cl. 705/3; 351/206; 705/2

(57) ABSTRACT

Systems and methods utilize computing devices to generate diagnoses of medical conditions. The computing devices can incorporate algorithms based on predetermined relationships between the medical conditions and various symptoms or characteristics associated with the medical conditions. The computing devices can prompt an individual evaluating patient information to observe whether the characteristics or symptoms are present in the patient information. The individual can subsequently provide inputs to the computing device indicating whether the characteristics or symptoms are present in the patient information.

### TECHNICAL FIELD

The embodiments and methods disclosed herein relate to practice of medicine, and have particular relevance to generating diagnoses of medical conditions in the practice of telemedicine.

### BACKGROUND

Telemedicine involves the diagnosis and potential management of medical conditions by a physician or other individual remotely located from the patient, using information transmitted by electronic means. Achieving consistent, accurate diagnoses is of primary importance in telemedicine, and in the practice of medicine in general. In particular, it is desirable to minimize physician to physician variations in diagnoses generated using the same or similar information. It is also desirable to minimize variations in diagnoses generated by the same physician at different times based on the same or similar information.

Achieving consistent, accurate diagnoses in the practice of telemedicine can be a challenge due to the lack of direct physical interaction between the patient and the physician. Moreover, the degree of inconsistency and inaccuracy in the diagnoses generated by a physician is generally higher when the physician does not have ready access to state of the art information and the current best practices in the physician's field of practice, a situation that can commonly occur in the practice of telemedicine.

Another issue in the practice of medicine is the lack of specialized physicians. For example, the availability of physicians specializing in the diagnosis and treatment of ocular diseases is generally less than the demand for such physicians, particularly in areas located away from major population centers.

Consequently, a need exists for systems and methods that can potentially enhance the consistency and accuracy of medical diagnoses, and that can help to maximize the efficiency of physicians or others in generating medical diagnoses.

### SUMMARY

Systems and methods utilize computing devices to generate diagnoses of medical conditions. The computing devices can incorporate algorithms based on predetermined relationships between the medical conditions and various symptoms or characteristics associated with the medical conditions. The computing devices can prompt an individual evaluating patient information to observe whether the characteristics or symptoms are present in the patient information. The individual can subsequently provide inputs to the computing device indicating whether the characteristics or symptoms are present in the patient information.

Methods comprise receiving medical information relating to a first individual; prompting a second individual to evaluate the medical information using a predetermined criterion; and generating a diagnosis of a medical condition based on a predetermined relationship between the results of the evaluation and the medical condition using a computing device.

Embodiments of computing devices comprise a processor, a memory communicatively coupled to the processor, and computer-executable instructions stored on the memory. The computing device receives medical information relating to a first individual; generates prompts for a second individual to evaluate the medical information using a predetermined criterion; and generates a diagnosis of a medical condition based on a predetermined relationship between the results of the evaluation and the medical condition.

Embodiments of systems comprise an imaging station comprising a first computing device, and an imaging device communicatively coupled to the first computing device. The embodiments also comprise a reading station comprising a second computing device, and a central data processing system. The central data processing system comprises a third computing device communicatively coupled to the first and second computing devices.

The third computing device receives images from the imaging station. The third computing device also generates prompts and sends the prompts and the images to the second computing device. The prompts prompt evaluation the images using a predetermined criterion.

The third computing device also receives information from the second computing device relating to the evaluation of the images, and generates a diagnosis of a medical condition based on a predetermined relationship between the information relating to the evaluation of the images and the medical condition.

### DRAWINGS

The foregoing summary, as well as, the following detailed description of preferred embodiments, are better understood when read in conjunction with the appended diagrammatic drawings. The drawings are presented for illustrative purposes only, and the scope of the appended claims is not limited to the specific embodiments shown in the drawings. In the drawings:

FIG. 1 is a diagrammatic illustration of a system for generating medical diagnoses;

FIG. 2 is a block diagram depicting a server of the system shown in Figure 1;

FIG. 3 is a block diagram depicting a computing device of an imaging station of the system shown in FIGS. 1 and 2;

FIG. 4 is a block diagram depicting a computing device of a reading station of the system shown in FIGS. 1-3;

FIGS. 5A and 5B are a flow diagram depicting a method for generating medical diagnoses;

FIG. 6 depicts a web page generated by the system shown in FIGS. 1-4, the web page prompting the input of various information relating to a patient;

FIG. 7 depicts another web page generated by the system shown in Figures 1-4, the web page displaying various locations on a patient's retinas at which images are to be acquired;

FIG. 8 depicts another web page generated by the system shown in Figures 1-4, the web page displaying a list of patients and related information associated with a particular reading physician using the system;

FIG. 9 depicts another web page generated by the system shown in Figures 1-4, the web page displaying a previously-acquired retinal image of one of the patients associated with the reading physician, and various observations of the retinal images to be made by the reading physician;

FIG. 10 depicts a web page generated by the system shown in FIGS. 1-4, the web page being capable of displaying a preliminary diagnostic report for review and approval of the reading physician;

FIG. 11 depicts a final report form generated using the system and method depicted in FIGS. 1-4, the final report being capable containing a diagnosis and recommendations relating to the presence of absence of retinal disease in a patient;

FIG. 12 depicts an algorithm for use in the system depicted in FIGS. 1-4, the algorithm being capable of generating diagnoses of diabetic retinopathy; and

FIG. 13 depicts another algorithm for use in the system depicted in FIGS. 1-4, the algorithm being capable of generating diagnoses of retinopathy of prematurity.

### DETAILED DESCRIPTION

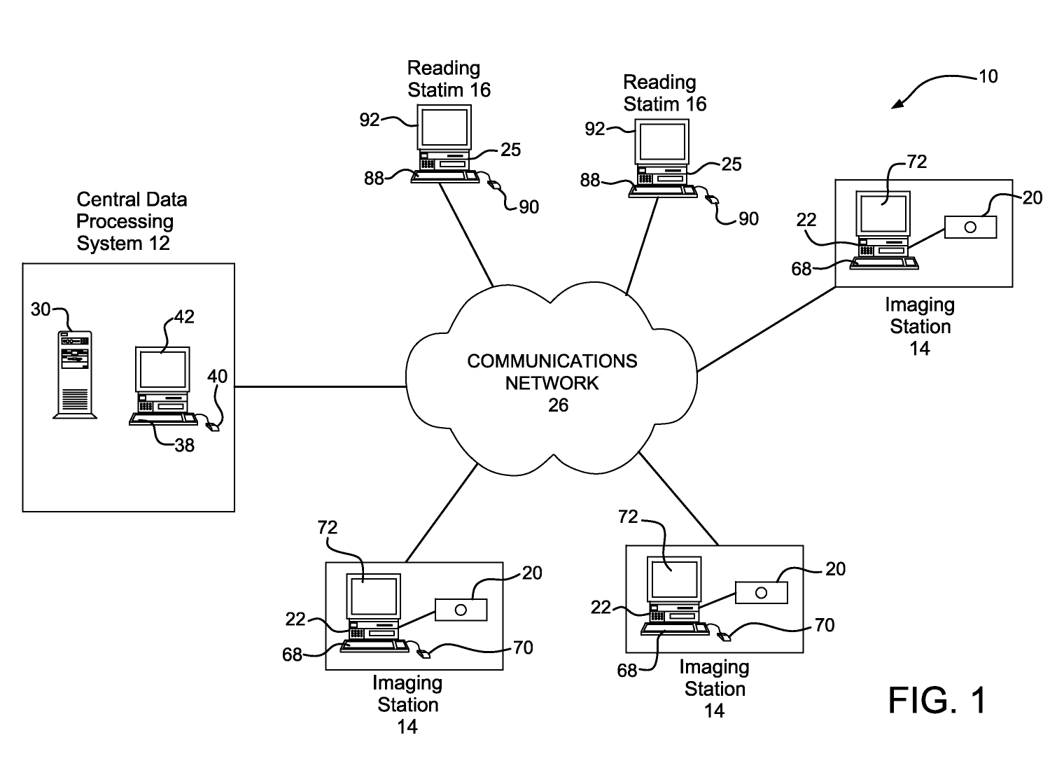

FIGS. 1 to 4 depict an embodiment of a system 10 for providing automated diagnoses of medical conditions. The system 10 comprises a central data processing system 12, one or more imaging stations 14, and one or more reading stations 16. As discussed below, the data system 12 is configured to generate a diagnosis of a medical condition such as retinal disease. The diagnosis is generated based on images captured by the imaging stations 14, and evaluations of the images made by a physician or other qualified individual at the reading stations 16.

The central data processing system 12, imaging stations 14, and reading stations 16 are depicted as being remotely located in relation to each other, i.e., as residing at different geographic locations. The central data processing system 12, and some or all of the imaging stations 14 and reading stations 16 can reside at the same location in alternative embodiments.

The use of the system 10 and the diagnostic methods described herein to diagnose various forms of retinal disease is described for exemplary purposes only. This particular application is disclosed for exemplary purposes only; the system 10 and the methods described herein can be adapted to diagnose other types of medical conditions, including medical conditions unrelated to the eyes.

The central data processing system 12, imaging stations 14, and reading stations 16 can communicate by way of a suitable communications network 26. The communications network 26 can be, for example, the internet, and the data processing system 12 can communicate with the imaging stations 14 and the reading stations 16 by way of a suitable protocol such as the hypertext transfer protocol (HTTP). The use of the internet as the communications network 26 is disclosed for exemplary purposes only; other suitable types of communications networks, such as a local area network, a wide area network, or an intranet, can be used in the alternative.

The system 10 is depicted with three of the imaging stations 14 for exemplary purposes only. Alternative embodiments can include less, or more than three imaging stations 14. The imaging stations 14 can each include an imaging device 20 suitable for acquiring images of the retina of a patient, as shown in FIG. 1. The imaging device 20 can be, for example, a digital camera such as a Nidek Fundus Camera.

Each imaging station 14 can also including a computing device 22 communicatively coupled to the imaging device 20. The computing device 22 can be any computing device, such as a desktop or notebook computer, capable of acquiring and storing digitized images from the imaging device 20, and transmitting the digitized images to the data processing system 12 by way of the communications network 26.

The system 10 is depicted with two of the reading stations 16 for exemplary purposes only. Alternative embodiments can include less, or more than three reading stations 16. Each reading station 16 can include a computing device 25 as shown in FIG. 1. Each computing device 25 is capable accessing the central data processing system 12 by way of the communications network 26. The computing device 25 can be any computing device, such as a desktop or notebook computer, capable of providing a means for a physician or other individual to interface with the central data processing system 12 via web pages generated and served by the central data processing system 12.

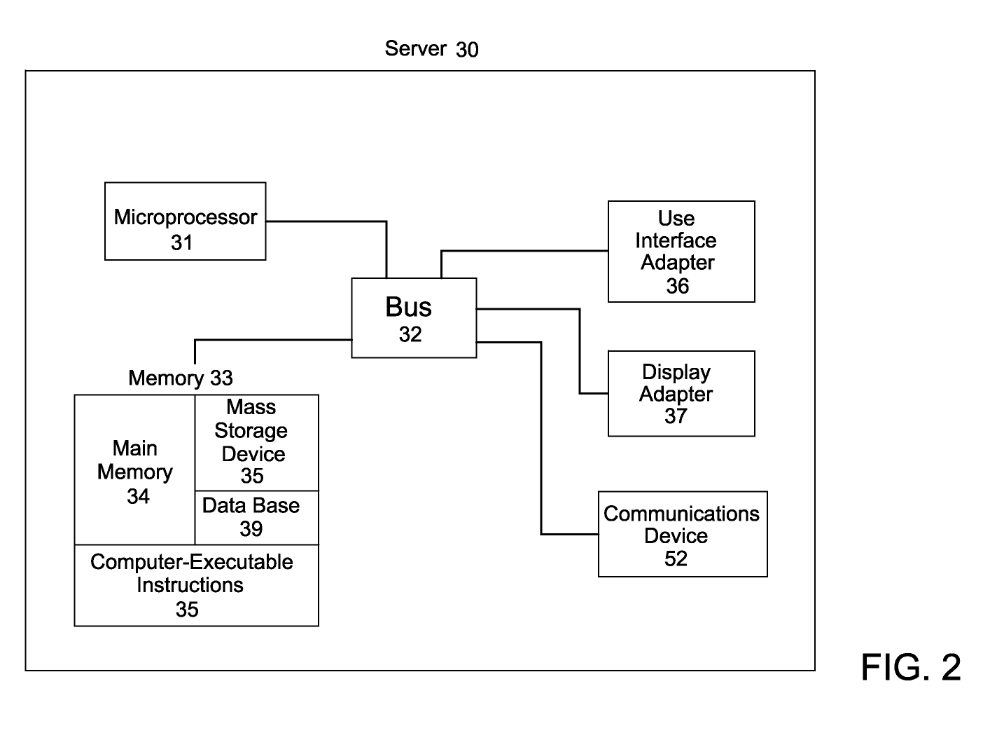

The central data processing system 12 can include a suitable computing device such as a server 30. The server 30 can comprise a processor such as a microprocessor 31, and a bus 32 that facilitates communication between the microprocessor 31 and various other components of the computing device 22, as shown in FIG. 2.

The server 30 can also include memory 33. The memory 33 can comprise a main memory 34 and a mass storage device 35, each of which is communicatively coupled to the microprocessor 31 by way of the bus 32. The main memory 34 can be, for example, random access memory. The mass storage device 35 can be, for example, a hard or optical disk.

The server 30 can also include computer-executable instructions 35 stored on the memory 33, as shown in FIG. 2. The computer-executable instructions 35, as discussed below, can generate diagnoses of various retinal diseases based on inputs received from the imaging stations 14 and the readings stations 16. Moreover, the computer-executable instructions 35 can include software that permits the server 30 to act as a web server.

The server 30 can also include a user interface adapter 36 and a display adapter 37 communicatively coupled to the microprocessor 31 by way of the bus 32. The server 30 can interface with the communications network 26 using a suitable communications device 52 such as a network card or modem.

The central data processing system 12 can include suitable interface devices that allow operators, programmers, or other individuals to interact with the server 30. For example, as shown in FIG. 1, the central data processing system 12 can include a keypad 38 and a mouse 40, each of which is communicatively coupled to the user interface adapter 36 of the server 30. The central data processing system 12 can also include a display device 42, such as a liquid crystal display (LCD) screen or monitor, communicatively coupled to the display adapter 37 of the server 30.

Specific details of the server 30 are provided for exemplary purposes only. Computing devices having hardware and software architecture other than that described above can be used in lieu of the server 30.

The use of a single server 30 in the central data processing system 12 is specified for exemplary purposes only. Alternative embodiments can be configured with multiple servers. For example, alternative embodiments can include a first server that functions as a web server; a second server used for data storage; and a third server used for processing the inputs from the imaging stations 14 in the manner discussed below.

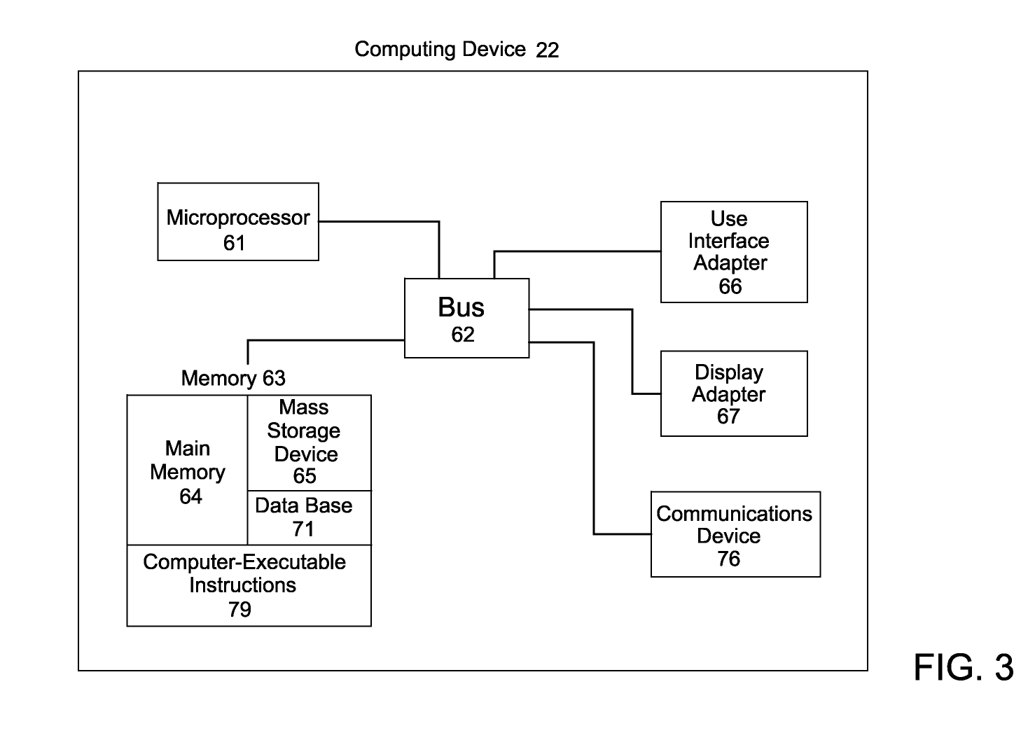

Each computing device 22 of the imaging stations 14 can include a processor such as a microprocessor 61, and a bus 62 that facilitates communication between the microprocessor 61, various other components of the computing device 22, and the imaging device 20 as shown in FIG. 3.

The computing device 22 can also include memory 63. The memory 63 can comprise a main memory 64 and a mass storage device 65, each of which is communicatively coupled to the microprocessor 61 by way of the bus 62. The main memory 64 can be, for example, random access memory. The mass storage device 65 can be, for example, a hard or optical disk.

Each computing device 22 can also include a user interface adapter 66 and a display adapter 67 communicatively coupled to the microprocessor 61 by way of the bus 62. The computing devices 22 can each interface with the communications network 26 and their corresponding imaging devices 20 using a suitable communications device 76 such as a network card or modem.

Each imaging station 14 can also include suitable interface devices that permit the technician or other individual operating the imaging device 20 to interact with the associated computing device 22. For example, as shown in FIG. 1, each imaging station 14 can include a keypad 68 and a mouse 70, each of which is communicatively coupled to the user interface adapter 66. The imaging stations 14 can each also include a display device 72, such as an LCD screen or monitor, communicatively coupled to the display adapter 67 of the corresponding computing device 22.

Each computing device 25 can include computer-executable instructions 79 that are stored on the memory 63 and executed on the microprocessor 61, as shown in FIG. 3. The computer-executable instructions 79 coordinate the display of the digital images and other information received from the imaging device 20, and the transmission of the images and information to the central data processing system 12.

The computer-executable instructions 79 can also include web browser software that permits the technician or other individual operating the imaging device 20 to view the web pages served to the computing device 22 by the server 30, and to initiate the transfer of the digital images and other information to the central data processing system 12.

Specific details of the computing devices 22 are provided for exemplary purposes only. Computing devices having hardware and software architecture other than that described above can be used in lieu of the computing devices 22.

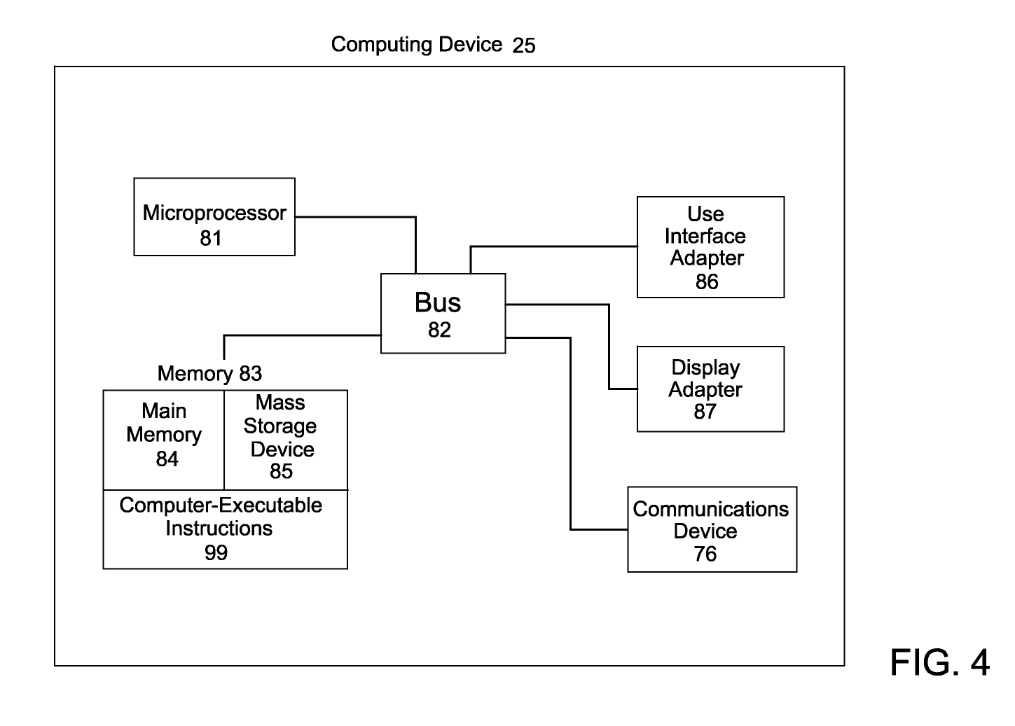

Each computing device 25 of the reading stations 16 can include a processor such as a microprocessor 81, and a bus 82 that facilitates communication between the microprocessor 81 and the various other components of the computing device 25 as shown in FIG. 4.

The computing device 25 can also include memory 83. The memory 83 can comprise a main memory 84 and a mass storage device 85, each of which is communicatively coupled to the microprocessor 81 by way of the bus 82. The main memory 84 can be, for example, random access memory. The mass storage device 85 can be, for example, a hard or optical disk.

Each computing device 25 can also include a user interface adapter 86 and a display adapter 87 communicatively coupled to the microprocessor 81 by way of the bus 82. Each computing device 25 can interface with the communications network 26 using a suitable communications device 76 such as a network card or modem.

Each reading station 16 can also include suitable interface devices that permit the physician or other individual evaluating the images displayed on the reading station 16 to interact with the associated computing device 25. For example, as shown in FIG. 1, each reading station 16 can include a keypad 88 and a mouse 90, each of which is communicatively coupled to the user interface adapter 86 of the corresponding computing device 25. The reading stations 16 can each also include a display device 92, such as an LCD screen or monitor, communicatively coupled to the display adapter 87.

Each computing device 25 can include computer executable instructions 99 that are stored on the memory 83 and executed on the microprocessor 81. The computer-executable instructions 99 can include web browser software 100 that permits the physician or other individual evaluating the retinal images to view the web pages served to the computing device 25 by the server 30 by the central data processing system 12, and to provide inputs to the central data processing system 12 based on the evaluations.

Specific details of the computing devices 25 are provided for exemplary purposes only. Computing devices having hardware and software architecture other than that described above can be used in lieu of the computing devices 25.

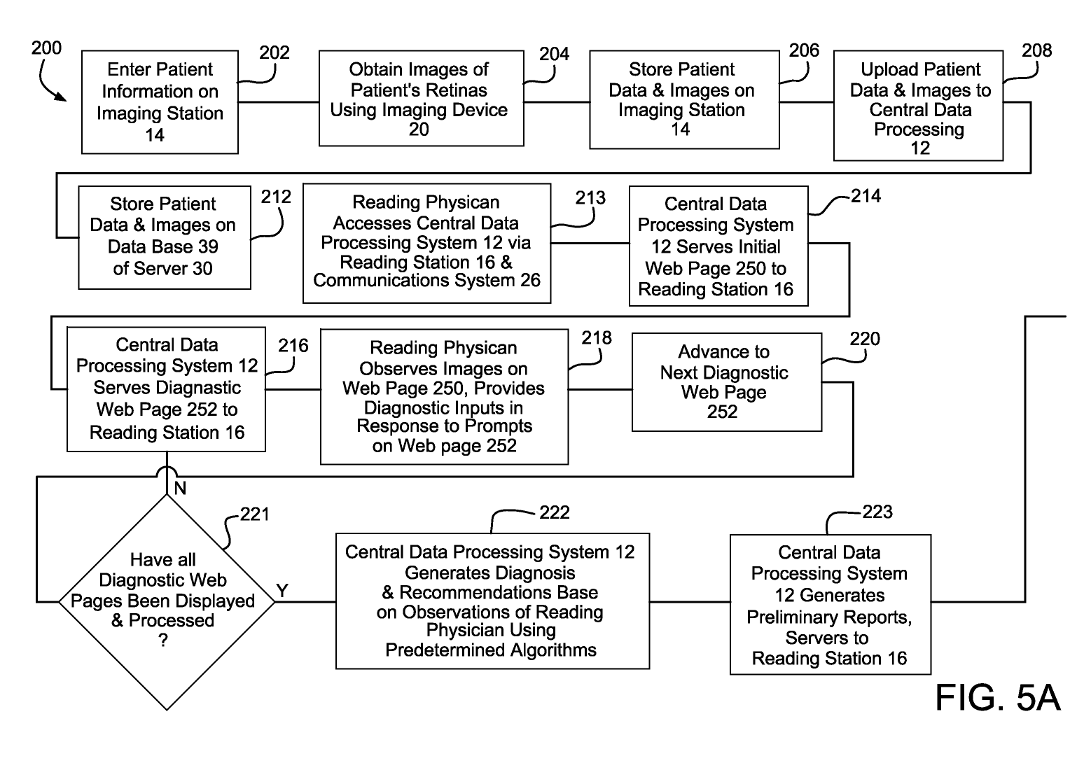

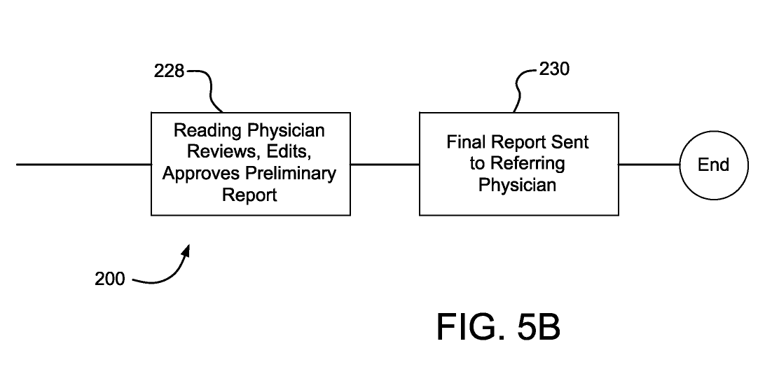

The system 10 can be used to diagnose retinal disease, such as diabetic retinopathy, in a patient in accordance with the exemplary method 200 depicted in FIG. 5.

When performing the method 200, one of the imaging stations 14 can be situated at a first location such as a mobile clinic or the office of a primary-care physician. The central data processing system 12 can be situated at a second location geographically remote from the first location. The reading station 16 can be situated at a third location, such as another physician's office, that is geographically remote from the first and second locations. The imaging station 14, central data processing system 12, and reading station 16 are described as being situated at three different locations for exemplary purposes only. The method 200 can be performed while the imaging station 14, the central data processing system 12, and/or the reading station 16 are co-located.

Images of the patient's retina can be obtained by a technician or other individual capable of operating the imaging device 20.

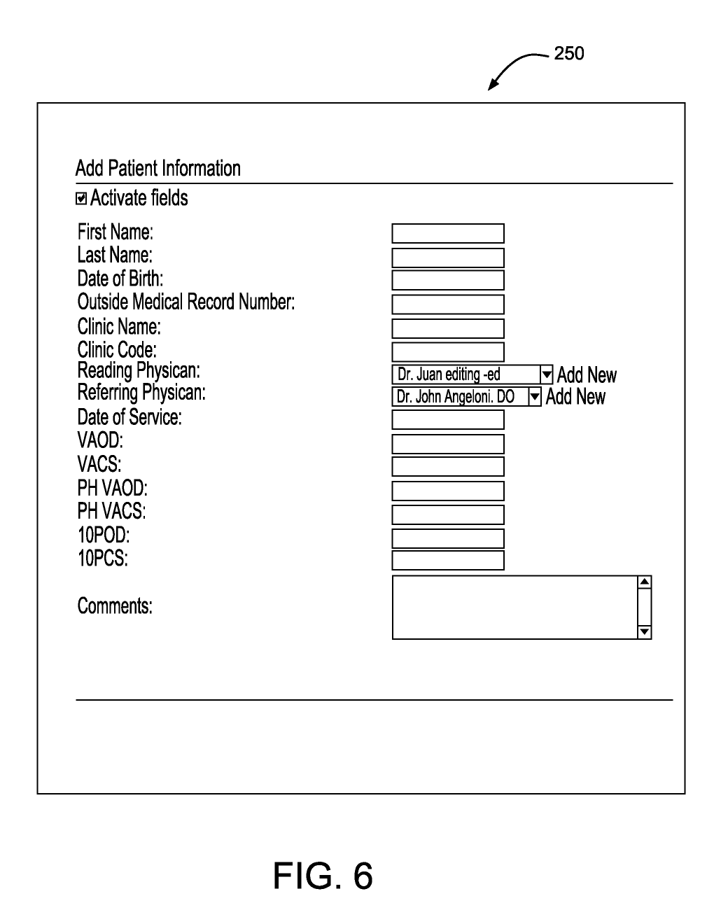

The computer-executable instructions 79 stored on the server 30 of the central data processing system 30 can cause a main web page (not shown) to be displayed on the display device 72. The main page can include an “Add Patient Information” prompt. The web page 250 depicted in FIG. 6 can be served by the server 30 and displayed on the display device 72 when the technician responds to the prompt. The technician can input information concerning the patient's identity using prompts provided by this screen (step 202 of FIG. 5). The technician can also enter information such as the type of diagnosis being made (such as diabetic retinopathy, macular degeneration, retinopathy of prematurity, etc.), and demographic and insurance-related information for the patient at this point.

The technician can subsequently obtain medical data in the form of images of the patient's retina, such as ocular fundus images or scanning ocular images, using the imaging device 20 (step 204). The type and number of images acquired are dependent upon the type of diagnosis that will subsequently be performed using the images. For example, a total of fourteen images, seven for each eye, can be obtained when screening for diabetic retinopathy. A total of twelve images, six for each eye, can be obtained when a screening for hypertension, macular degeneration, and glaucoma is being conducted. A total to twenty-four images, twelve for each eye, can be obtained when a full diabetic retinal examination is being performed. Instructions concerning the type of examination can be provided to the technician by, for example, a physician who has referred the patient for the examination.

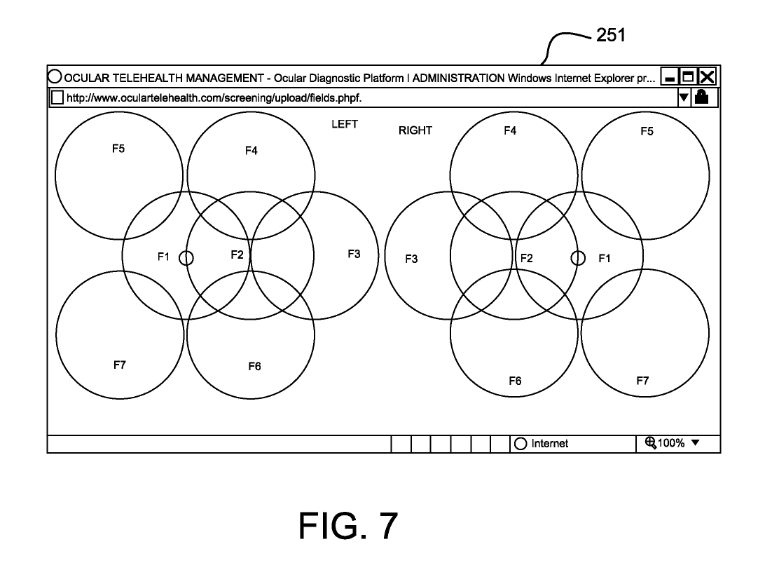

The central data processing system 12 can provide guidance concerning the retinal images to be acquired by the technician. For example, the server 30 can serve the web page 251 shown in FIG. 7 to the imaging station 14. The web page 251 provides a graphical depiction of the specific locations at which retinal images are to be acquired for a screening for hypertension, macular degeneration, and glaucoma.

The retinal images and corresponding patient information can be stored on a data base 71 residing in the memory 63 of the computing device 22 (step 206). The retinal images and patient information can subsequently be uploaded to the server 30 of the central data processing system 12 by way of the communications network 26, using a suitable protocol such as file transfer protocol (FTP) (step 208). The information can be designated in the data base 71 as “uploaded” after the information has been successfully transmitted to the server 30.

The images and patient information can be uploaded at the end of each work day, along with images and information for other patients acquired during that day. The images and patient information can be uploaded at other times, e.g., immediately after the images have been acquired, in the alternative. The images and information can be deleted from the computing device 14, for example, after a predetermined amount of time has elapsed, or when the memory storage space is subsequently needed for additional images and information.

The retinal images and patient information transmitted to the central data processing system 14 from the computing device 22 can be stored in a data base 39 residing on the sever 30 of the central data processing system 14 (block 212). The images and patient information can subsequently be viewed and evaluated by an individual with sufficient training, such as a ophthalmologist (hereinafter referred to as a “reading physician”), using predetermined prompts and other guidance provided by the central data processing system 12.

The reading physician can be located at a location different than the location of the central data processing system 12, and can access the central data processing system 12 via one of the reading stations 16 and the communications network 26 (step 213 of FIG. 5). In particular, the reading physician can use the web browser of the computing device 25 of the reading station 16 to access the web site hosted by the server 30 of the central data processing system 12. The reading physician can access an account stored on the data base 39 by, for example, inputting a user ID and a password in response to a prompt displayed on a home page (not shown) generated and served by the server 30.

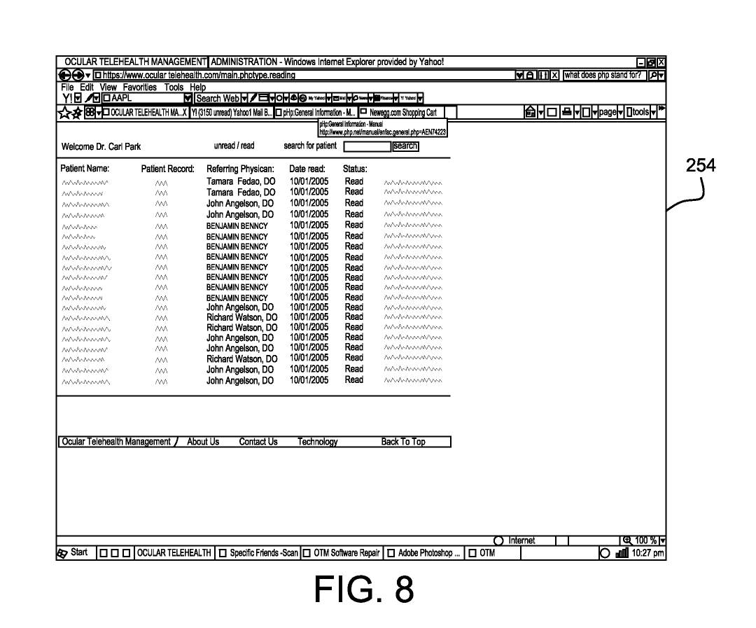

The server 30 serves an initial web page 254 depicted in FIG. 8 when the reading physician accesses his account (step 214). The initial web page 254 can include a list of all of the patients listed in the data base and associated with the reading physician. The initial web page 254 can also include information such as the name of the referring physician corresponding to each patient; the status (read or unread) of the data associated with each patient; the date on which the patient data was evaluated by the reading physician (if applicable); etc. The reading physician can view the initial web page 254 on the display device 92 of the reading station 16, and can select a particular patient from the list of patients using the web browser of the computing device 25.

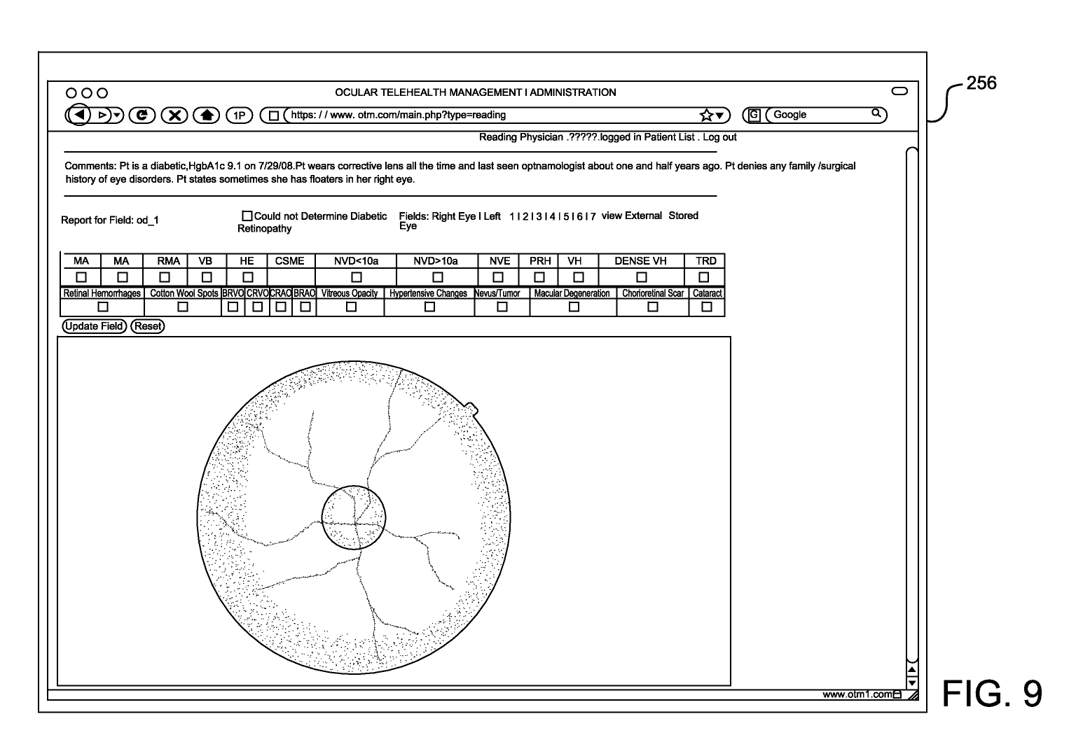

Upon selection of a particular patient by the reading physician, the server 30 serves the diagnostic web page 256 shown in FIG. 9 (step 216). The diagnostic web page 256 includes one of the images of the patient's retinas acquired previously acquired and stored in the data base 39 on the server 30. The diagnostic web page 256 also includes text that indicates various symptoms or conditions of the retina that should be evaluated by the reading physician when viewing the image, in view of the particular diagnosis that is being made. The computer-executable instructions 35 of the server 30 can be configured to automatically select and serve a particular set of diagnostic web pages corresponding to the particular diagnosis being made, based on the patient information sent to the reading station 16.

The symptoms and conditions associated with a particular diagnosis can be chosen, for example, based on state of the art and/or the current best practices in the relevant medical field, e.g., retinal opthamology. The symptoms and conditions can be chosen, for example, by one or more physicians or scientists considered to be an authority in the field.

A box appears on the diagnostic web page 556 adjacent to the text denoting the symptoms or conditions that should be evaluated, as shown in FIG. 9. The reading physician can use the web browser of the computing device 25 to check the box if the condition or symptom described by the corresponding text is observed by the reading physician when viewing the retinal image being displayed (step 218). The text displayed on the diagnostic web page 256 thus prompts the reading physician to make certain observations regarding the image, and to provide inputs indicative of what the reading physician has observed by checking or not checking the corresponding boxes.

For example, the diagnostic web page 256 depicted in FIG. 9 prompts the reading physician to make observations relevant to a diagnosis of diabetic retinopathy. As indicated in FIG. 9, the physician is prompted to determine whether the following symptoms or conditions are visible in each image: microaneurism only (MA); microaneurism/hemorrhage (HMA); intraretinal microvascular abnormality (IRMA); venous beading (VB); hard exudate (HE); clinically significant macular edema (CSMA); neovascularization of disc (NVD<10a, NVD>10a); neovascularization elsewhere (NVE); preretinal hemorrhage (PRH); vitreous hemorrhage (VH); vitreous hemorrhage with no retinal details (DENSE VH); traction retinal detachment (TRD), etc. The reading physician checks a box located next to each symptom or condition if the reading physician observes the condition or symptom in the image.

The particular conditions and symptoms listed on the diagnostic web page 256 are dependent upon, and will vary with the specific type of retinal disease or other medical condition being diagnosed.

Once the reading physician has made each of the prompted observations and provided the corresponding inputs, the reading physician can advance to the next image in the set of retinal images for the patient by responding to an “advance” prompt on the initial web page 252 (step 220). Another diagnostic web page 256 containing the next image is served by the server 30 and appears on the display device 92 of the reading station 16 at this point. This diagnostic web page 256 also includes text prompting the reading physician to make various observations of the image, and boxes that the reading physician can check based on the observations.

The above process can be repeated until the reading physician has observed and provided inputs concerning each of the images in the set of images for the patient (step 221).

The reading physician can subsequently prompt the server 30 to generate a diagnosis and recommendations by using the web browser to click on a tab labeled “interpretation” on the web page containing the final image in the set. The diagnosis and recommendations are generated by algorithms incorporated into the computer-executable instructions 35 (step 222). The algorithms are capable of interpreting each possible combination of checked and unchecked boxes for the series of retinal images as corresponding to a particular diagnosis and an associated recommendation.

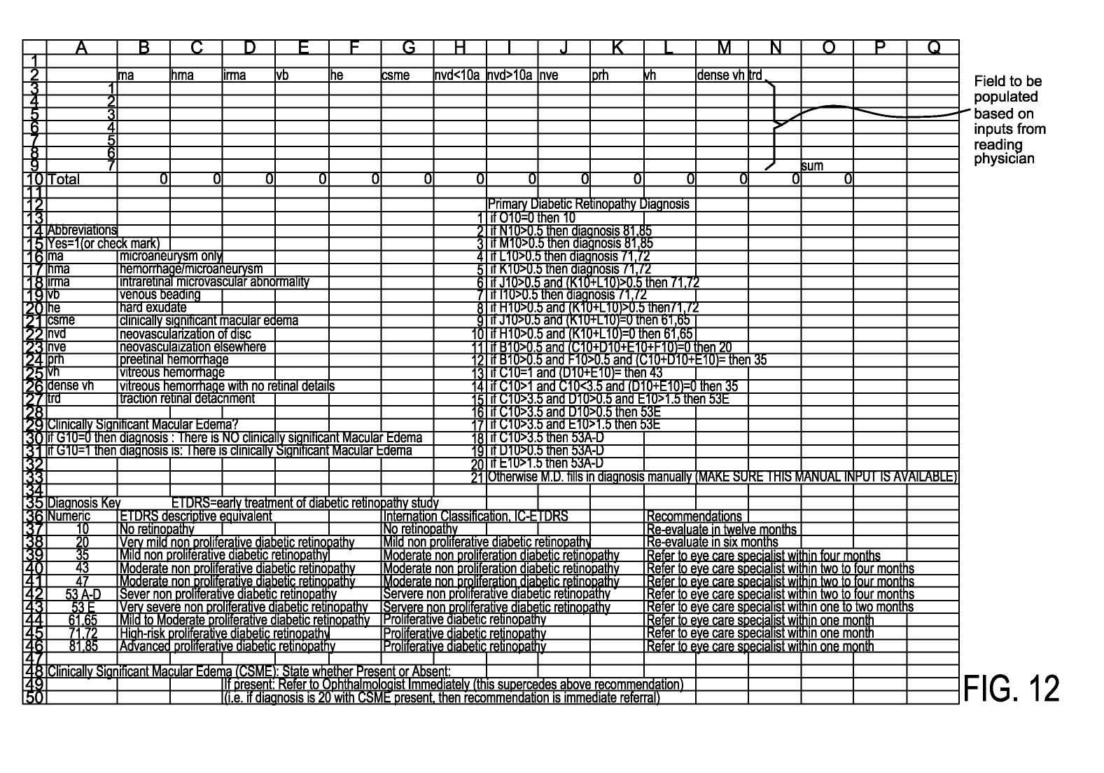

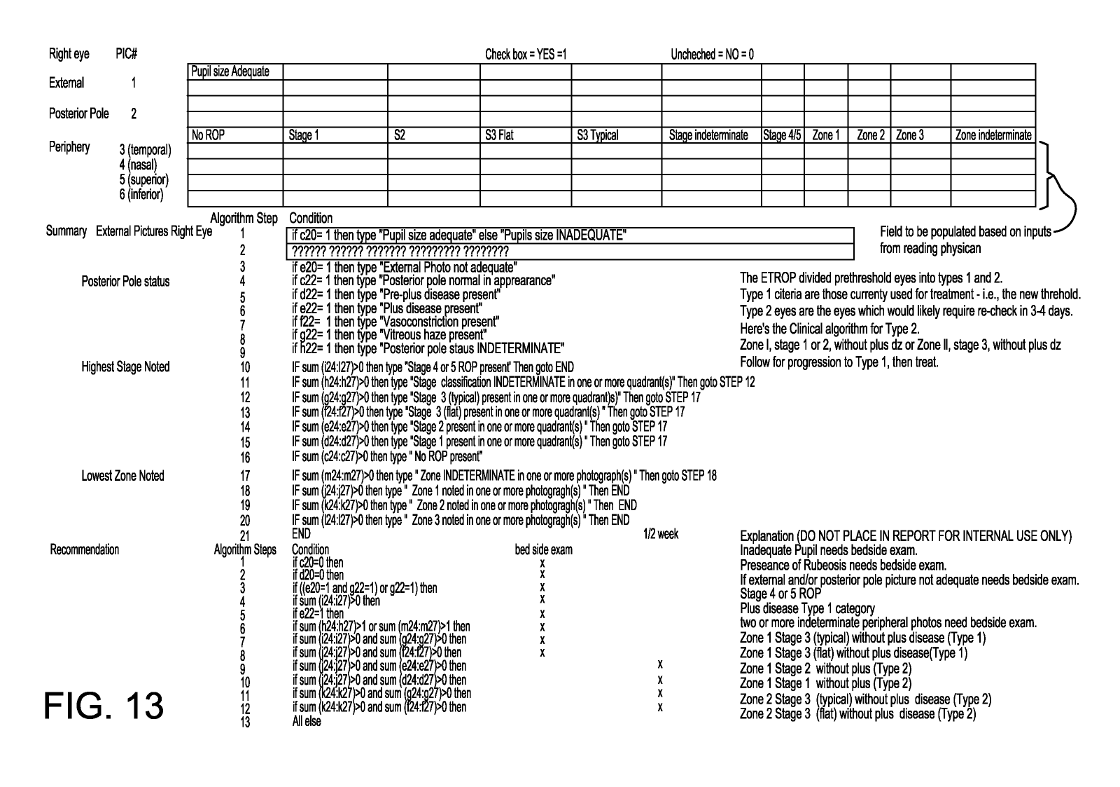

FIG. 12 depicts an exemplary algorithm that can be used to generate diagnoses and corresponding recommendations relating to diabetic retinopathy, based on the observations prompted by the diagnostic web pages 256 corresponding to each of the seven images acquired for each eye. FIG. 13 is an exemplary algorithm that can be used in the alternative to the algorithm depicted in FIG. 12, to generate diagnoses of retinopathy of prematurity based on observations prompted by an associated diagnostic web page (not shown).

The algorithms, and the above-noted observations that are used as inputs to the algorithms, can be generated based on inputs from one or more physicians, scientists, or individuals considered to be authorities in the relevant field, and can be updated on an as-needed and/or periodic basis. This helps to ensure that the diagnoses produced using the algorithms reflect the state of the art and the current best practices in the corresponding medical field.

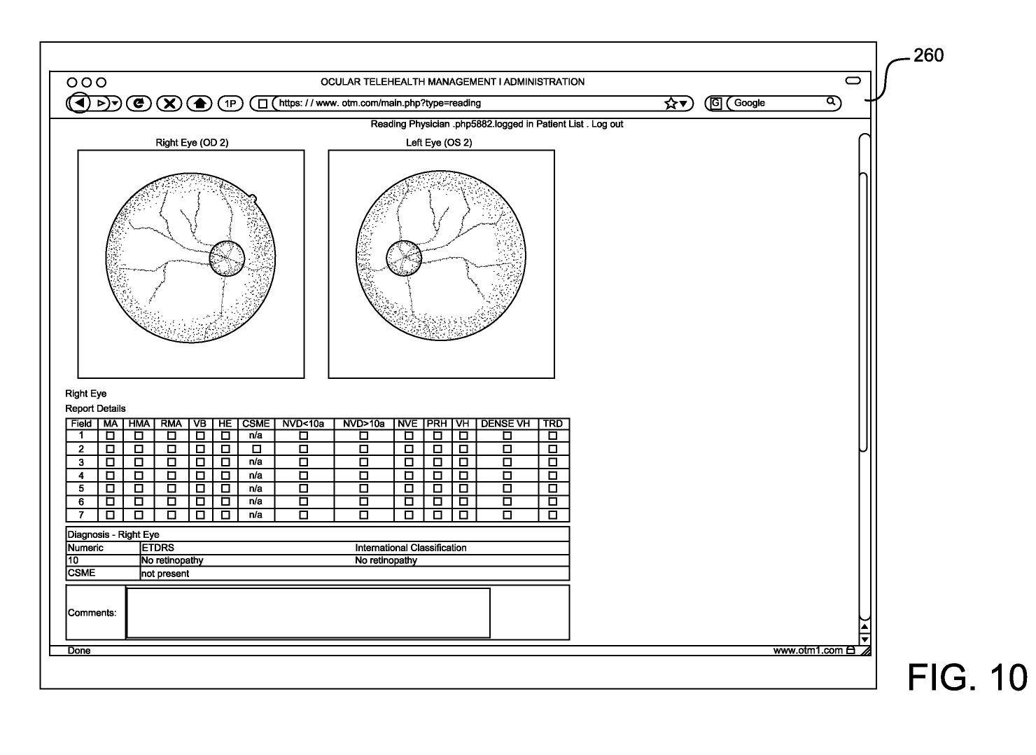

The computer-executable instructions 35 can be configured to cause the server 30 to generate a series of web page 260 containing a preliminary report. A blank, i.e., not yet filled in, preliminary report is depicted in FIG. 10. The preliminary report contains the findings of the diagnostic process, the diagnosis, and the recommendations (step 223). The preliminary report can be accessed by the reading physician from the reading station 16, and can be displayed on the display device 72. The web pages 260 can be served automatically, immediately after the preliminary report is generated. Alternatively, the web pages 260 can be served in response to a prompt generated by the reading physician from the reading station 25.

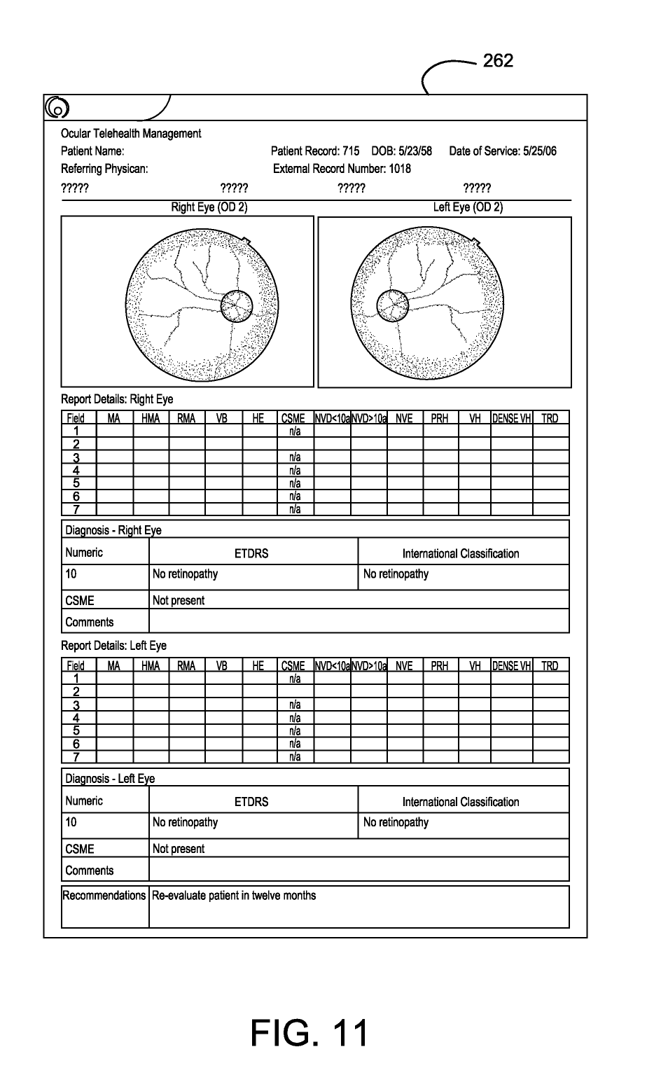

The reading physician can review, edit, and approve the report (step 228 of FIG. 5). The reading physician can approve the report by clicking on a tab on the report labeled “approve,” using the web browser of the computing device 25. A final report 262 is subsequently generated and saved to the data base 39, the corresponding patient data is marked as “read,” and the web page listing the patients associated with the reading physician and having unread data in the data base is once again displayed on the display device 92 (step 230). A blank final report is depicted in FIG. 11.

The final report 262 can be sent to the referring physician in electronic or paper form (step 230). The final report 262 can sent on an automatic basis, immediately after being generated. Alternatively, a system administrator can send the final report 262 at a later time, e.g., at the end of the day together with other reports. The final report 262 can be sent using a suitable means such as e-mail, fax, regular mail, or overnight courier, pre-selected by the referring physician.

The computer-executable instructions 35 can also be configured to cause the central data base 12 to send billing charges and information a third party billing system. The transmission of billing information can occur automatically, or upon an input of the system administrator. The billing information, including information relating to the patient's health insurance, can be stored in the data base 39.

The computer-executable instructions 35 can be configured to facilitate general maintenance of the system 10, and to monitor the status of the server 30, the health of the database 39, and usage of the system 10. These features can be implemented using, for example, MYSQL® open source software.

The computer-executable instructions 35 of the server 30 can be configured to cause the server 30 to generate reminders for referring physicians and/or patients concerning follow up examinations; and reports relating to the frequency of use of the system 10 by each referring physician. The computer-executable instructions 35 can also be configured to cause the server 30 to generate summary reports for the reading physicians. These summary reports can include, for example, information relating to the transmission of the reports generated by the reading physician, the number of reports generated by the reading physician per session or per hour, and payments made to the reading physician.

The computer-executable instructions 35 can also be configured to cause the server 30 to generate invoices for reading physicians, technicians, usage fees, etc. The reminders, reports, invoices, etc. can be sent electronically via e-mail or another suitable electronic medium; alternatively, hard copies can be generated and sent via regular mail or courier.

The algorithms embedded in the computer-executable instructions 35, as discussed above, can cause the server 30 to generate diagnoses of medical conditions by processing the observations in accordance with the state of the art and/or best practices in the relevant medical field. Moreover, the algorithms can be updated as the state of the art and/or best practices evolve or otherwise change. The diagnoses generated using the system 10 and method 200 can thus be based on the best, most up-to-date medical information available at the time the diagnoses are made.

Moreover, diagnoses generated using the system 10 and the method 200 can have relatively high levels of accuracy, repeatability, and reliability due to the use of a pre-determined set of algorithms that (i) prompt the reading physician like a checklist to make specific observations concerning the medical data acquired from the patient, and (ii) generate diagnoses based on the observations. The algorithms can thus help to compensate for the lack of direct physical interaction between the reading physician and the patient.

It is believed that the use of the system 10 and method 200 can make the diagnostic process more efficient by reducing the time and effort needed to generate diagnoses for each patient. A reading physician can thus generate diagnoses for a larger number of patients during a given time frame than would otherwise be possible. Moreover, by prompting observations and automatically generating diagnoses based on the observations, the system 10 and method 200 can potentially allow an individual with a comparatively low degree of education or training, e.g., a nurse or a physician's assistant, to generate diagnoses that would otherwise have to be generated by an individual with a higher degree of training, e.g., a physician. The use of the system 10 and method 200 can thus potentially increase the availability of medical care to patients that otherwise would not have access to such care.

The foregoing description is provided for the purpose of explanation and is not to be construed as limiting the invention. Although the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Those skilled in the relevant art, having the benefit of the teachings of this specification, can make numerous modifications to the invention as described herein, and changes may be made without departing from the scope and spirit of the invention as defined by the appended claims.

What is claimed is:

1. A method, comprising:
receiving medical information relating to a first individual;
prompting a second individual to evaluate the medical information using a predetermined criterion; and
generating a diagnosis of a medical condition based on a predetermined relationship between the results of the evaluation and the medical condition using a computing device.

2. The method of claim 1, further comprising receiving information relating to the evaluation.

3. The method of claim 1, wherein receiving medical information relating to a first individual comprises receiving medical information relating to an eye of the first individual.

4. The method of claim 3, wherein receiving medical information relating to an eye of the first individual comprises receiving information representing an image of a retina of the eye.

5. The method of claim 1, wherein prompting a second individual to evaluate the medical information using a predetermined criterion comprises prompting the second individual to make an observation of an image generated using the medical information relating to a first individual.

6. The method of claim 5, wherein prompting the second individual to make an observation of an image generated using the information relating to a first individual comprises prompting the second individual to determine whether a specific condition and/or symptom is visible in the image.

7. The method of claim 6, wherein:
receiving medical information relating to a first individual comprises receiving information representing a retinal image of an eye of the individual; and
prompting the second individual to determine whether a specific condition and/or symptom is visible in the image comprises prompting the second individual to determine whether one of more of the following conditions or symptoms is visible in the retinal image: microaneurism only; microaneurism/hemorrhage; intraretinal microvascular abnormality; venous beading; hard exudate; clinically significant macular edema; neovascularization of disc; neovascularization elsewhere; preretinal hemorrhage; vitreous hemorrhage; vitreous hemorrhage with no retinal details; and traction retinal detachment.

8. The method of claim 1, wherein generating a diagnosis of a medical condition based on a predetermined relationship between the results of the evaluation and the medical condition using a computing device comprises diagnosing hypertension; macular degeneration; and/or glaucoma using the computing device.

9. The method of claim 1, wherein generating a diagnosis of a medical condition based on a predetermined relationship between the results of the evaluation and the medical condition using a computing device comprises using a server to generate the diagnosis of the medical condition.

10. The method of claim 1, wherein generating a diagnosis of a medical condition based on a predetermined relationship between the results of the evaluation and the medical condition using a computing device comprises using a computing device comprising computer-executable instructions that incorporate algorithms representing the predetermined relationship between the results of the evaluation and the medical condition.

11. The method of claim 1, wherein prompting a second individual to evaluate the medical information using a predetermined criterion comprises causing text identifying a particular symptom or characteristic of the medical condition to be displayed to the second individual.

12. The method of claim 2, wherein receiving information relating to the evaluation comprises receiving information concerning whether the second individual has observed the particular symptom or characteristic in the medical information.

13. The method of claim 1, further comprising sending the medical information to the second individual using the computing device; and wherein prompting a second individual to evaluate the medical information using a predetermined criterion comprises generating prompts using the computing device.

14. The method of claim 1, wherein the second individual is a physician specializing is diagnosing the medical condition.

15. The method of claim 1, wherein the first individual is located at a first location and the second individual is located at a second location remote from the first location.

16. The method of claim 15, wherein the computing device is located at third location remote from the first and second locations.

17. A computing device, comprising a processor, a memory communicatively coupled to the processor, and computer-executable instructions stored on the memory, wherein the computing device receives medical information relating to a first individual; generates prompts for a second individual to evaluate the medical information using a predetermined criterion; and generates a diagnosis of a medical condition based on a predetermined relationship between the results of the evaluation and the medical condition.

17. The computing device of claim 16, wherein the computing device prompts the second individual to make an observation of an image generated using the medical information relating to a first individual.

18. The computing device of claim 17, wherein the computing device prompts the second individual to determine whether a specific condition and/or symptom is visible in the image.

19. The computing device of claim 18, wherein: the computing device receives information representing a retinal image of an eye of the individual, and prompts the second individual to determine whether one of more of the following conditions or symptoms is visible in the retinal image: microaneurism only; microaneurism/hemorrhage; intraretinal microvascular abnormality; venous beading; hard exudate; clinically significant macular edema; neovascularization of disc; neovascularization elsewhere; preretinal hemorrhage; vitreous hemorrhage; vitreous hemorrhage with no retinal details; and traction retinal detachment.

20. The computing device of claim 16, wherein the computing device generates a diagnosis of hypertension; macular degeneration; and/or glaucoma.

21. The computing device of claim 16, wherein computing device is a server.

22. The computing device of claim 16, wherein the computer-executable instructions incorporate algorithms representing the predetermined relationship between the results of the evaluation and the medical condition.

23. The computing device of claim 16, wherein the computing device prompts the second individual to evaluate the medical information using a predetermined criterion by causing text identifying a particular symptom or characteristic of the medical condition to be displayed to the second individual.

24. The computing device of claim 16, wherein computing device generates a report containing the diagnosis.

25. A system, comprising:
an imaging station comprising a first computing device, and an imaging device communicatively coupled to the first computing device;
a reading station comprising a second computing device; and
a central data processing system comprising a third computing device communicatively coupled to the first and second computing devices, wherein the third computing device:
receives images from the imaging station;
generates prompts and sends the prompts and the images to the second computing device, the prompts prompting evaluation the images using a predetermined criterion;
receives information from the second computing device relating to the evaluation of the images; and
generates a diagnosis of a medical condition based on a predetermined relationship between the information relating to the evaluation of the images and the medical condition.

26. The system of claim 25, wherein the imaging device is a camera.

27. The system of claim 25, wherein the first, second, and third computing devices are communicatively coupled by way of a communications network.

28. The system of claim 27, wherein the communications network is the internet.

29. The system of claim 25, wherein the information from the second computing device relating to the evaluation of the images is generated in response to the prompts.

30. The system of claim 25, wherein the third computing device comprises computer-executable instructions that incorporate algorithms representing the predetermined relationship between the predetermined relationship between the information relating to the evaluation of the images and the medical condition.

31. The system of claim 25, wherein the imaging station is located at a first location and the reading station is located at a second location remote from the first location.

32. The method of claim 31, wherein the central data processing system is located at third location remote from the first and second locations.
